SEQUENCE LISTING (6 sequences)

SEQ ID NO: 1 - DNA, 2240 bp, Homo sapiens
ttccaagctggttttgaagtcgcggcagctgttcctgggacgtccggttgaccgcgcgtctgctgcagagaccatgtctgccgacggggcagaggctgatggcagcacccaggtgacagtggaagaaccggtacagcagcccagtgtggtggaccgtgtggccagcatgcctctgatcagctccacctgcgacatggtgtccgcagcctatgcctccaccaaggagagctacccgcacatcaagactgtctgcgacgcagcagagaagggagtgaggaccctcacggcggctgctgtcagcggggctcagccgatcctctccaagctggagccccagattgcatcagccagcgaatacgcccacagggggctggacaagttggaggagaacctccccatcctgcagcagcccacggagaaggtcctggcggacaccaaggagcttgtgtcgtctaaggtgtcgggggcccaagagatggtgtctagcgccaaggacacggtggccacccaattgtcggaggcggtggacgcgacccgcggtgctgtgcagagcggcgtggacaagacaaagtccgtagtgaccggcggcgtccaatcggtcatgggctcccgcttgggccagatggtgttgagtggggtcgacacggtgctggggaagtcggaggagtgggcggacaaccacctgccccttacggatgccgaactggcccgcatcgccacatccctggatggcttcgacgtcgcgtccgtgcagcagcagcggcaggaacagagctacttcgtacgtctgggctccctgtcggagaggctgcggcagcacgcctatgagcactcgctgggcaagcttcgagccaccaagcagagggcacaggaggctctgctgcagctgtcgcaggccctaagcctgatggaaactgtcaagcaaggcgttgatcagaagctggtggaaggccaggagaagctgcaccagatgtggctcagctggaaccagaagcagctccagggccccgagaaggagccgcccaagccagagcaggtcgagtcccgggcgctcaccatgttccgggacattgcccagcaactgcaggccacctgtacctccctggggtccagcattcagggcctccccaccaatgtgaaggaccaggtgcagcaggcccgccgccaggtggaggacctccaggccacgttttccagcatccactccttccaggacctgtccagcagcattctggcccagagccgtgagcgtgtcgccagcgcccgcgaggccctggaccacatggtggaatatgtggcccagaacacacctgtcacgtggctcgtgggaccctttgcccctggaatcactgagaaagccccggaggagaagaagtagggggagaggagaggactcagcgggccccgtctctataatgcagctgtgctctggagtcctcaacccggggccatttcaaacttattttctagccactcctcccagctcttctgtgctgtccacttgggaagctaaggctctcaaaacgggcatcacccagttgacccatctctcagcctctctgagcttggaagaagcctgttctgagcctcaccctatcagtcagtagagagagatgtccagaaaaaatatctttcaggaaagttctcccctgcagaattttttttccttgttaaatatcaggaatataggccgggtgcggtggctcacacctgtaatcccagcactttgggaggctgaggcgggcggaacacctgaggtcaggtgttcgagaccagccaggccaacatggtgaaaccccgtctctactaaaaatacaaaaaaaaatgagccgggcatggtagcaggtgtctgttatcccagttaggaggctgaggcaagagaatctcttgaacctgagaggcggaggttgcagtgagccaagatcgcgccattgcactccagcctgggggacaagagtgagacttagtctcaaaaaaaaaaaaaaagaaaaaaaaatcagggatatagttcatatcccacttctttgtttacaccgatgtccctgaatatcagcctgtagctaatggacttgggatttctggtctaagtgggcctcctggggatggggtggtacactgagcttctgagcctcattgtagagtagaaaggtactggggcctgtgtggtaagccttgttgaaatgctctggtattcagtattgccttaataaacttcacccacaactgcatacaggcaaaaa

SEQ ID NO: 2 - protein, 434 aa, Homo sapiens
MSADGAEADGSTQVTVEEPVQQPSVVDRVASMPLISSTCDMVSAAYASTKESYPHIKTVCDAAEKGVRTLTAAAVSGAQPILSKLEPQIASASEYAHRGLDKLEENLPILQQPTEKVLADTKELVSSKVSGAQEMVSSAKDTVATQLSEAVDATRGAVQSGVDKTKSVVTGGVQSVMGSRLGQMVLSGVDTVLGKSEEWADNHLPLTDAELARIATSLDGFDVASVQQQRQEQSYFVRLGSLSERLRQHAYEHSLGKLRATKQRAQEALLQLSQALSLMETVKQGVDQKLVEGQEKLHQMWLSWNQKQLQGPEKEPPKPEQVESRALTMFRDIAQQLQATCTSLGSSIQGLPTNVKDQVQQARRQVEDLQATFSSIHSFQDLSSSILAQSRERVASAREALDHMVEYVAQNTPVTWLVGPFAPGITEKAPEEKK

SEQ ID NO: 3 - DNA, 21 bp, Artificial Sequence
  sense primer
agagatggtgtctagcgccaa

SEQ ID NO: 4 - DNA, 22 bp, Artificial Sequence
  anti-sense primer
cggtcactacggactttgtctt

SEQ ID NO: 5 - DNA, 29 bp, Artificial Sequence
  sense primer
taatacggatccatgtctgccgacggggc

SEQ ID NO: 6 - DNA, 30 bp, Artificial Sequence
  anti-sense primer
taagtcgagctcctacttcttctcctccgg

(19) United States
(12) Patent Application Publication
(10) Pub. No.: US 2011/0033865 A1
(43) Pub. Date: Feb. 10, 2011

(54) NOVEL METHOD FOR DIAGNOSING PREGNANCY-RELATED COMPLICATIONS

(75) Inventors: Hamutal Meiri, Tel Aviv (IL); Marei Sammar, Tamra (IL); Moran Sade, Karmiel (IL)

Correspondence Address: THE NATH LAW GROUP, 112 South West Street, Alexandria, VA, 22314, US

(73) Assignee: DIAGNOSTIC TECHNOLOGIES LTD., Yoqneam (IL)

(21) Appl. No.: 12/937,622

(22) PCT Filed: Apr. 5, 2009
(86) PCT No.: PCT/IL09/00366

§ 371 Date: Oct. 13, 2010

Related U.S. Application Data
(60) Provisional application No. 61/071,209, filed on Apr. 17, 2008.

(51) Int. Cl. C12Q 1/68; G01N 33/53; C12Q 1/02

(52) U.S. Cl. 435/6; 436/501; 435/29; 436/86; 435/7.21

(57) ABSTRACT

A method for diagnosing pregnancy-related complications in a pregnant woman is provided. The method includes the following steps: (a) determining the level of Placental Protein 17 (PP17) in a bodily substance obtained from the pregnant woman; and (b) comparing the determined level of PP17 to a standard level of PP17, a significant modification in the level of PP17 indicating the existence of a pregnancy-related complication in the pregnant woman. A diagnostic kit is also described.

### FIELD OF THE INVENTION

This invention relates to a method for diagnosing pregnancy-related complications in a pregnant woman.

### BACKGROUND OF THE INVENTION

The goal of pregnancy management is the delivery of a mature, healthy infant, without encountering complications which can adversely affect the well being of both the mother and the newborn. A significant percentage of pregnancies are affected by various disorders. Among them are preterm delivery, intrauterine growth retardation and preeclampsia. These complications negatively impact the outcome of affected pregnancies, at enormous cost both to the patients as well as to the health system.

Pregnancy-related proteins are proteins produced during pregnancy by either the mother, the fetus or the fetoplacental unit. In certain cases, some of these proteins may be used to monitor the intactness of the pregnancy. For example, U.S. Pat. No. 5,198,366 describes a RIA for detecting Placental Protein 13 (PP13) as a diagnostic tool for pregnancy-related complications.

Bohn, H. (1983) Oncodev. Biol. Med. 4:343-350 describes the isolation and physico-chemical characterization of a soluble placental protein named Placental Protein 17 (PP17). PP17 was not observable in any fetal or adult organ extracts other than placenta.

Than, N. G., et al (1998) Eur. J. Bioch. 258:752-757 describes the cloning and sequence analysis of cDNAs encoding human PP17 variants. Monospecific anti-PP17 rabbit serum was used to detect four PP17 immunoreactive proteins in term placental tissue extract: (1) PP17a (31500 kDa); (2) PP17b (48000 kDa); (3) PP17c (60900 kDa); and (4) PP17d (74,000 kDa). PP17c was found to be a dimmer. An elevation in serum levels of PP17 variants during healthy pregnancy was noted.

Than, N. G., et al (1999) Tumor Biol. 20:184-192 describes the isolation of cDNAs encoding PP17a from a human placental cDNA library. The PP17 protein was found to have sequence homology with adipophilin and the mouse adipose differentiation-related protein. PP17b was found to be identical with the protein Tail Interacting Protein 47 (TIP47), and to be involved in apoptotic and differentiation processes of human epithelial cervical carcinoma cells (Than, N. G., et al (2003) Eur. J. Bioch. 270:1176-1188).

The full consensus nucleotide sequence of PP17b is found under the gene bank accession number NM 005817 as presented in FIG. 1, and the amino acid sequence of PP17b is found under the NCBI protein accession number NP 005808 as presented in FIG. 2. The wild type PP17b is composed of 434 amino acids.

The entire contents of all of the above references are hereby incorporated by reference.

### SUMMARY OF THE INVENTION

In one aspect, the present invention provides a method for diagnosing pregnancy-related complications in a pregnant woman comprising: (a) determining the level of Placental Protein 17 (PP17) in a bodily substance obtained from said pregnant woman; and (b) comparing the determined level of PP17 to a standard level of PP17, a significant modification in the level of PP17 indicating the existence of a pregnancy-related complication in said pregnant woman.

The following are further embodiments of the invention:
The method of the invention wherein the bodily substance is selected from placental tissue and body fluids from maternal blood, maternal saliva, maternal urine, amniotic fluid, umbilical cord blood and chorionic
The method of the invention wherein the level of PP17 is determined by measuring the level of PP17 DNA.
The method of the invention wherein the DNA is cDNA.
The method of the invention wherein the level of PP17 is determined by measuring the level of PP17 RNA.
The method of the invention wherein the PP17 mRNA expression level is measured.
The method of the invention wherein the level of PP 17 is determined by measuring the level of PP17 protein.
The method of the invention wherein the level of PP17 protein is determined using an immunoassay.
The method of the invention wherein the pregnancy-related complication is preeclampsia.
The method of the invention combined with at least one additional method for diagnosing pregnancy-related complications in a pregnant woman.
A kit for diagnosing pregnancy-related complications in a pregnant woman comprising:
   a first antibody which specifically binds to PP 17;
   a second antibody which specifically binds to PP 17 linked to a signal-generating molecule; and
   PP17 standard solutions.
A kit for diagnosing pregnancy-related complications in a pregnant woman comprising:
   specific PP17 primers; and
   positive and negative cDNA controls.

In the context of the present specification, the term “PP17” includes all variants of PP17, including PP17a, PP17c, PP17d, and in particular PP17b.

A “standard level of PP17” is defined as the level of PP17 in the corresponding tissues of a healthy pregnant woman who delivered a healthy baby in term (=normal pregnant woman). The standard level may be based on the level of PP17 from a previous healthy pregnancy of the same woman. Preferably, the level of PP17 is measured at a corresponding stage of the pregnancy (e.g. 1^{st} trimester, 24^{th} week, etc.). At times, the terms “standard level” and “normal level” are used interchangeably.

The level of PP17 can vary as a function of time (gestational weeks), as a function of the genetic and physical characteristics of the woman such as body mass index, maternal age, ethnicity, and parity, and as a function of the identity of the bodily substance measured. Therefore, when comparing a measured PP17 value from a patient to the standard level of PP17, these parameters should be taken into account. At times, the measured PP17 value will be normalized in order to compare it to the corresponding standard level of PP17.

Pregnancy-related complications include all of the various types of diseases and disorders which may affect a pregnant woman as a result of her pregnancy, as listed, for example, in the Merck Manual (e.g. Chapters 252 and 253 in the 17^{th} edition (1999)). In particular, the term refers to preeclampsia. In the present specification, the term “preeclampsia” (PE) includes all types of the disease, including mild, severe, early onset, late onset, PE complicated by intrauterine growth restriction (IUGR), and HELLP (hemolysis, elevated liver enzymes and low platelet count), unless specifically indicated otherwise.

A bodily substance may include any fetal or adult tissue which contains PP17 (see for example No. 2202122 [Mannose-6-phosphate receptor binding protein-1=PP17] as tested and published by GNF—Genome Institute of Novartis Research Foundation). PP17 is particularly abundant in the following tissues: adipocyte, bronchial epithelial cells, PB-BDCA4± dendritic cells, BM CD34+, placenta, prostate, testis, 721-B-Lymphoblasts, PB-CD14-Monocyte, cardiomyocytes, smooth muscles, BM-CD33+myeloid, thymus and thyroid. In particular, the term includes placental tissue and body fluids from maternal blood, maternal saliva, maternal urine, amniotic fluid, umbilical cord blood and chorionic villi.

In the present invention, the term “determining” includes both qualitative as well as quantitative determinations. The method of the invention may determine the current presence of the disease in the woman, and/or a predisposition of the woman to the disease.

The term “significant” as in a “significant increase” between standards and samples, is defined, in cases of a qualitative comparison, as a difference of 20% or more, preferably 30% or more, more preferably 40% or more, most preferably 50% or more, between the measured values of the standard and the sample, all other parameters, remaining the same. For a quantitative comparison, it is defined as a statistical difference between standards and samples with P<0.05, preferably P<0.001 or lower for the comparison of the differences.

The term “modification” means a significant change in the determined level of PP17 from the standard level. The change may be either an increase or a decrease in the level of PP17. In one embodiment, the modification is an increase.

PP17 may be obtained from a pregnant woman in a number of ways. Non-limiting examples include:
a purified preparation from body fluids, particularly amniotic fluid;
PP17-encoding DNA isolated from human placenta and expressed in host cells or in a cell-free preparation;
after purification from the placenta or other tissue sources, or from maternal placenta derived primary cultures or immortalized cell lines;
by chorionic villous sampling (CVS) or their derived placenta tissue cultures or cultured trophoblasts; and
from miscarriage or abortion tissues.

The level of PP17 may be determined by various techniques. For example, PP17 mRNA expression may be determined or PP17 protein levels may be measured.

In order to measure PP17 mRNA expression levels, quantitative real time PCR and/or normal PCR assays may be used. Such assays and diagnostic kits for carrying out such assays are further aspects of the invention. The steps of the assay may include the following:
RNA preparation;
generation of cDNA;
amplification of PP17 cDNA by specific primers; and
quantification of the PP17 level as compared to a reference gene.

In one embodiment, the invention provides a kit for diagnosing pregnancy-related complications in a pregnant woman comprising:

(a) specific PP17 primers; and

(b) positive and negative cDNA controls.

Examples of positive and negative cDNA controls may be placenta tissue and brain tissue, e.g. globus palidus nuclei, respectively.

Additional optional components which may be included in the kit include:

(c) Reverse transcriptase;

(d) sFree DNTP;

(e) TAQ Polymerase.

In order to measure PP17 protein levels, immunoassays such as dot blot, Western blot and enzyme immunoassays may be used. Such assays and diagnostic kits for carrying out such assays are further aspects of the invention. In one non-limiting example, the assay may include the following components:
monospecific polyclonal or monoclonal first anti-PP17 antibodies coupled to a solid phase support;
a PP17 standard (either recombinant PP17 or native PP17 purified by biochemical procedures);
a second polyclonal or monoclonal anti-PP17 coupled to a ligand (e.g. biotin); and
a corresponding ligand (e.g. avidin, strepavidin or any other avidin derivate) coupled to a signal-generating molecule (e.g. HRP, AP-etc.).

The detection of the signal could be colorimetric, chemiluminescent, delphia or other platforms.

The steps of the assay may include the following:
coupling first anti-PP17 antibodies to a solid phase support;
incubating the sample containing PP17 or a control with the support;
adding a second polyclonal or monoclonal anti-PP17 coupled to a ligand;
adding the corresponding ligand coupled to a signal-generating molecule;
measuring the signal produced by the signal-generating molecule as compared to the signal generated by the control.

Thus, in another embodiment, the invention provides a kit for diagnosing pregnancy-related complications in a pregnant woman comprising:

(a) an antibody which specifically binds to PP17;

(b) a second antibody linked to a signal-generating molecule; and

(c) PP17 standard solutions.

### BRIEF DESCRIPTION OF. THE DRAWINGS

In order to understand the invention and to see how it may be carried out in practice, embodiments will now be described, by way of non-limiting example only, with reference to the accompanying drawings, in which:

FIG. 1 shows the complete nucleotide sequence of PP17b (Accession no. NM 005817). The first ATG and the termination codon (TAG) are underlined (SEQ ID NO:1);

FIG. 2 shows the amino-acid sequence of PP17b (SEQ ID NO:2);

FIG. 3 shows an SDS-PAGE Code stained gel (A) and a Western blot gel (B) illustrating PP17 expression in the absence (−) and presence (+) of d-isopropyl-β-D-thiogalactoside (IPTG);

FIG. 4 shows SDS-PAGE gels illustrating PP17 expression and purification. Generation of recombinant PP17 is based on cloning of the PP17 gene in a pQE30 expression vector, with a 6-His-tag. Escherichia Coli strain M15 (pREP4) were used as a host cell, transformed in PP17 cloned in a pQE30 vector. The rPP17 was induced by a special induction medium including IPTG, followed by protein purification by Ni-NTA agarose. Bound rPP17 was eluted from the agarose with Imadizole. Samples from each step of purification were analyzed by SDS-PAGE;

| Gel lane | FIG. 4A | FIG. 4B |
|---|---|---|
| 1 | Molecular weight markers | |
| 2 | Supernatant (original) | |
| 3 | Pellet (original) | Elution 6 |
| 4 | Flow through | Elution 7 |
| 5 | Wash | Elution 8 |
| 6 | Elution 1 | Elution 9 |
| 7 | Elution 2 | Elution 10 |
| 8 | Elution 3 | Elution 11 |
| 9 | Elution 4 | |
| 10 | Elution 5 | |

FIG. 5 shows photographs of a PCR gel of PP17 in-term placentas taken from healthy and diseased women as compared to a reference protein (tyrosine 3-monooxygenase/tryptophan 5-monooxygenase activation protein, zeta polypeptide=YWHAZ); and

FIG. 6 is a log 2 scale relative expression ratio plot of PP17 gene in PE (A) and HELLP (B) patients as compared to normal control (normal placenta).

### DETAILED DESCRIPTION OF EMBODIMENTS

### Materials and Methods

Cohort: Patients delivered at the Bnai-Zion Hospital, Haifa, Israel, and at the Wolfson Medical Center, Holon, Israel, were recruited to the study after signing an informed consent, and their placenta were collected after delivery. The cohort was comprised of 9 preeclamptic (PE) patients delivered before 37 weeks (1 before 34 weeks), 3 HELLP patients delivered before 37 (1 delivered before 34 weeks), 5 preterm delivery (PTD) patients delivered before 37 weeks (3 before 34 weeks) and 9 normal pregnant women delivered at term (>37 weeks). Preeclampsia was diagnosed according to hypertension of 140 (systolic) or 90 (diastolic) mmHg developed after 20 weeks of gestation in women who were normotensive before and accompanied by proteinuria of 2+in a dipstick or 300 mg/dL at 24 hr collection in women with no protein in the urine before pregnancy. HELLP was diagnosed as preeclampsia plus two of the following a-c: a) hemolysis (lactic dehydrogenase>600 IU/l, or serum bilirubin>1.2 mg/dl, or the presence of schistocytes in the peripheral blood); b) increased serum aspartate aminotransfarase concentration (>=70 IU/l); c) thrombocytopenia (platelet count<100,000/mm3). PTD were women who delivered before 37 weeks not due to preeclampsia or IUGR but idiopathic preterm delivery.

Table 1 shows the summary of clinical characteristics of the patient and control groups (Values are Mean±standard deviation).

TABLE 1
Clinical characteristic of patients

| | Preeclampsia | HELLP | Normal |
|---|---|---|---|
| Characteristics | N = 9 | N = 3 | controls (N = 9) |
| Age (yrs) | 26.8 ± 3.98 | 30.66 ± 4.93 | 32.5 ± 3.9 |
| Gestational age | 30.63 ± 2.55 | 28 ± 1 | 38.1 ± 1 |
| (wks) | | | |
| Primiparas | 5/9 (55.5%) | 1/3 (33.3%) | 2/9 (22.2%) |
| Birth weight | 1278 ± 406 | 847 ± 88.9 | 3538 ± 379 |
| (gr) | | | |

RNA Extraction and First Strand cDNA Synthesis:

Total RNA was extracted from 100 mg placental tissue using Trizol Reagent (Invitrogen). RNA concentration and purity were monitored by spectrophotometer at 260/280 nm (Beckman Coulter) and stored in DNase/RNase-free water until use. The first strand cDNA was synthesized with SuperScript II RT reverse transcriptase using a Oligo(dT)15 (Promega) as follow: 3 μg of total RNA were reverse transcriptized by 200 units of Superscript RT using 25 ng of oligo(dT)15 and 0.5 mM of dNTP mixture at 42° C. for 50 min.

Quantitative Real Time PCR:

The sense primer 5′-AGAGATGGTGTCTAGCGCCAA-3′ (SEQ ID NO:3), and anti-sense primer 5′-CGGTCACTACGGACTTTGTCTT-3′ (SEQ ID NO:4) (primerBank ID 20127486a3) were used for real time PCR to amplify PP17 using QPCR SYBER Green (Abgene™, UK) on Gene Amp 5700 sequence detection system (Applied Biosystem, USA). PCR was performed with 2μl cDNA, 10μl of x2 QPCR SYBER Green mix and 2 μl of sense and antisense primers (70 nM each). PCR was conducted by one 15 min cycle at 95° C. and 40 cycles of two-steps of 15 sec at 95° C. and 1 min at 60° C. Samples were normalized to the level of the YWHAZ determined in parallel. Following amplification, melt curves were generated to confirm the specificity of each primer pair. The fold increase relative to placenta of a control case was determined by 2^{-ΔΔCt} method and relative expression software tools-REST (References: (1) Pffafl M. W. A new mathematical model for relative quantification in rel-time RT-PCR, Nucleic Acids Res. (2001), 29; 2001-2007 (2) Pffafl MW et al: Relative expression software tool (rest) for group-wise comparison and statistical analysis of relative expression results in real time PCR, Nucleic Acids Res. (2002), 30; e36). PCR products were further analyzed using 2% agarose gels and visualized with ethidium bromide.

### EXAMPLES

### Example 1

### Cloning the Full Length PP17 Gene, Expression in Host Cells and Affinity Purification of Recombinant PP17 (rPP17)

1—Poly Chain Reaction—PCR

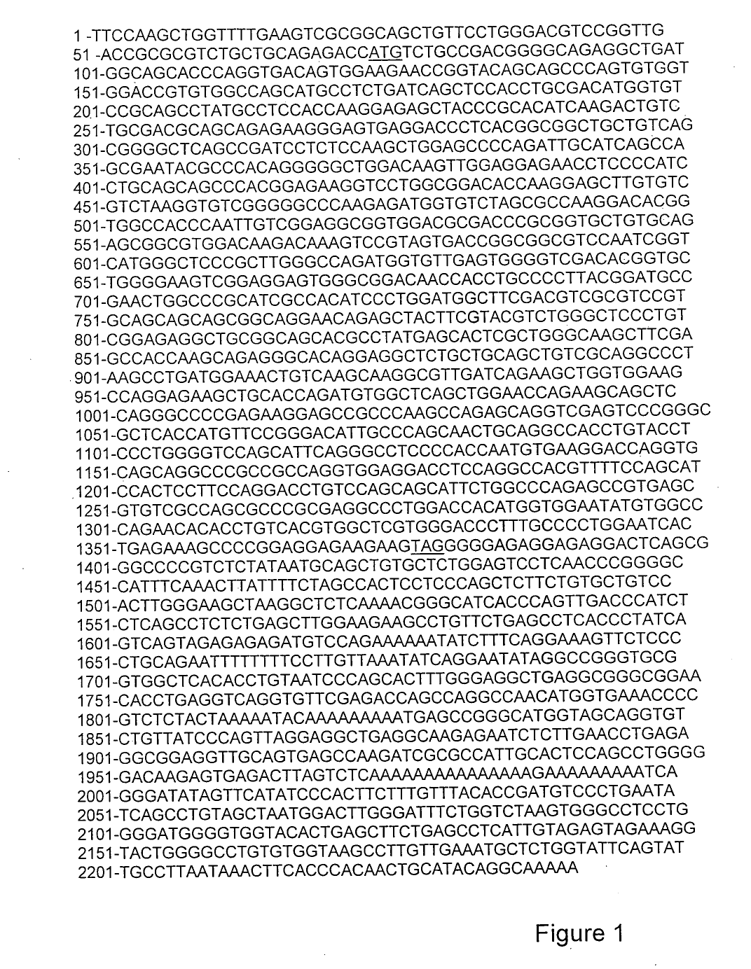

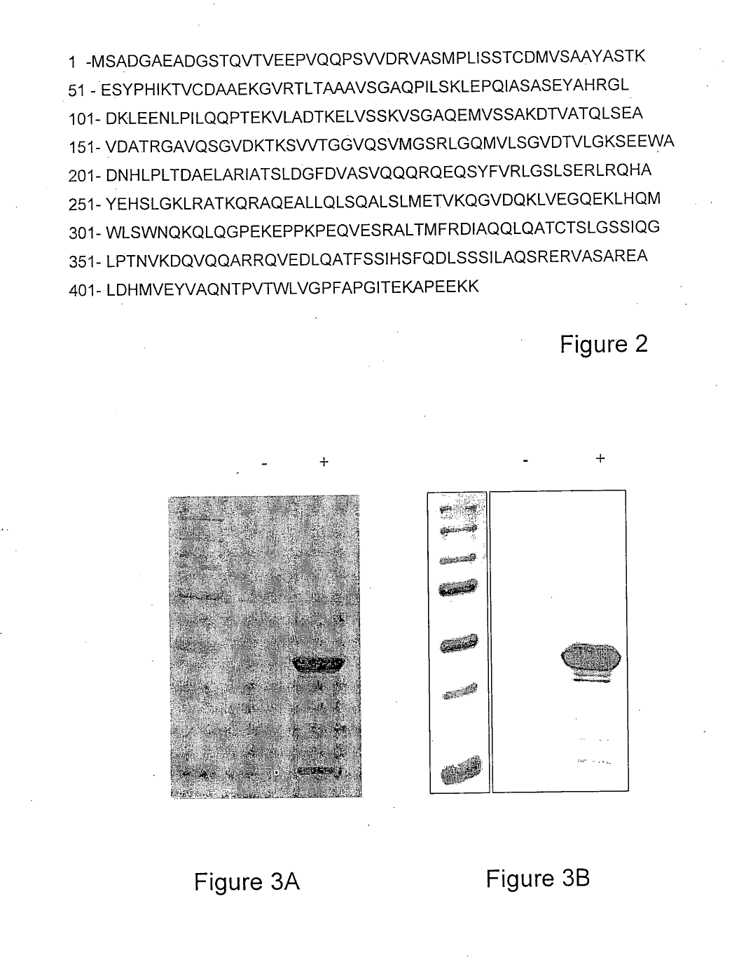

The nucleotide sequence of PP17 with accession no. NM 005817 as shown in FIG. 1 was used for selection and design of two primers to cover the full length of the gene. For cloning the full-length PP17 gene, two primers were designed with the following sequences:

sense primer: 5′-TAATACGGATCCATGTCTGCCGACGGGGC-3′ (SEQ ID NO:5) and anti-sense primer: 5′-TAAGTCGAGCTCCTACTTCTTCTCCTCCGG-3′ (SEQ ID NO:6). The restriction site sequences of BamH I and Sac I were introduced into the sense and antisense primers, respectively. Both primers were synthesized by Sigma-Genosys. 1 ng of PP17 cDNA was amplified with 0.2 μM of the above mentioned specific primers using the Red PCR Master Mix containing Tage DNA polymerase, dNTP's and MgCl2 (LAROVA). PCR was carried out under the following conditions: 94° C. for 2 min once, then 94° C. for 30 sec, 55° C. for 30 sec and 72° C. for 2 min over 35 cycles. A final extension was carried out at 72° C. for 4 min followed by storage of the PCR product at 4° C. until use. The PCR product was analyzed by agarose gel and revealed the expected size of 1.3 kb.

2—Cloning of the DNA into Expression Vector.

a—Ligation: The PCR product of PP17 DNA was purified by QIAquick PCR purification kit prior to ligation. The expression vector pQE-30 was purchased from Qiagen. Both the pQE-30 (0.5 μg) and the purified PCR product DNA (1 μg) were digested with BamH I and Sac I (20 U each) in NEBuffer BamH I and NEBuffer Sac I, respectively. Both enzymes were purchased from New England Biolabs (NEB). An insert:vector ratio of 3:1, 1:1 and 1:3 was used for ligation of the digested PCR product DNA with 50 ng of digested pQE-30 using 100 U of T4 ligase (NEB) and T4 ligase buffer for 2 hr at 22° C.

b—Transformation: The ligation mixture was transferred to M15 (pREP4) cells (Qiagen). 10 μl of the ligation mixture were added to 100 μl Competent M15 pREP4 cells for 10 min in ice and then transferred to a 42° C. water bath for 50 sec. After heat shock, the mixture was placed in ice for another 2 min and 900 μl of LB medium were added to the transformation reaction and incubated for 60 min at 37° C. with shaking of approximately 225 rpm. 10-100 μl of the cells were plated on LB agar plate containing 100 μg/ml ampicillin (Sigma) and 25 μg/ml Kanamycin (Sigma) for overnight at 37° C.

c—Screening for Positive Colonies: 20 single colonies grown on the plate were chosen and cultured in 2 ml LB medium containing ampicillin (100 μg/ml) and kanamycin (μg/ml) overnight at 37° C. with 225 rpm shaking. Plasmid DNA was purified from each colony culture with Wizard Plus SV minipreps DNA purification system (Promega). The presence of the PP17 DNA insert was tested by PCR as follow: the PCR reaction (20 μl volume) was composed of 1 ng of DNA template, 0.2 μM of PP17 specific primers (SEQ. ID. NOS. 5 and 6) and 10 μl of x2 ready mix for PCR (Bio-Lab Ltd). The PCR conditions were as described above. PCR products were separated on 1% agarose and the DNA bands were visualized in a LAS-3000 image system (Fuji). The potential positive clones (4) were selected according to the calculated size of the PCR product. The final DNA sequence of each clone was determined by sequencing carried out in the multi-disciplinary laboratories unit (Rappaport Institute Of Medical Science—Technion, Haifa).

3—Expression of the Recombinant PP17

1. Based on sequence analysis, one bacterial positive clone carrying PP17 cloned in pQE30 vector was selected for expression of the protein and grown in 20 ml of LB medium containing ampicillin and Kanamycin at 37° C. for overnight with shaking. The culture was mixed 1:50 in LB medium containing antibiotics and grown at 37° C. until an OD₆₀₀ of 0.6 was reached. The expression of the protein was induced with 1 mM—isopropyl-b-D-thiogalactopyranoside (IPTG) for 3 hrs at 37° C. (FIG. 3). Bacterial cells were harvested by centrifugation at 4000 g×20 min at 4° C. The cell pellet was stored until use at −80° C. Aliquots were collected before and after induction and PP17 was tested by SDS-PAGE followed by Gel Code staining (3A) and by Western blot (3B) using anti-histidine antibodies and visualized by LAS3000 system to test PP17 expression as a recombinant protein.

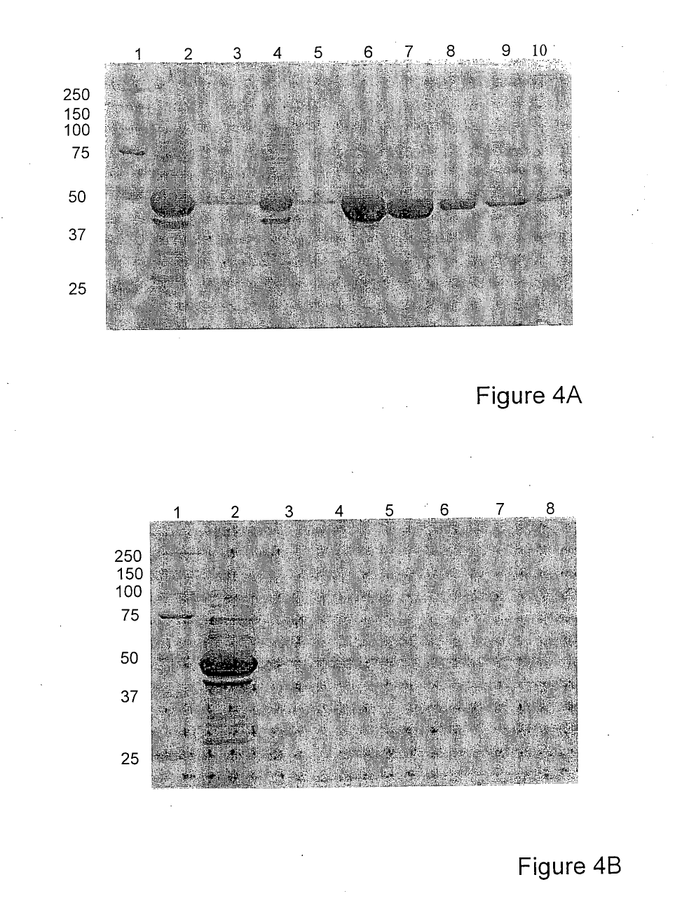

2. Purification of PP17: Based on SDS-PAGE analysis, the recombinant PP17 was localized in the soluble fraction. The cell pellet was resuspended in lysis buffer containing 20 mM Tris-HCl, pH 8, 150 mM NaCl, 5 mM imadizole and protease inhibitor (Roche) and 10% glycerol. The cells were disrupted by applying pressure of 1000 PSi in minicell French press (Thermo). Soluble proteins were collected and the pellet containing the inclusion bodies was discarded. Soluble fraction was filtered through 0.25 μm filters and mixed with 1 ml of pre-equilibrated Ni-NTA agarose (Qiagen) for 1 hr at RT. First the Ni-NTA agarose column was washed with 20 ml of wash buffer (20 mM Tris-HCl, pH 8.0, 300 mM NaCl, 20 mM Imidazole, PMSF, Complete and 10% glycerol). Bound recombinant PP17 was eluted with 15 fractions of 1 ml of elution buffer (20 mM Tris-HCl, pH 8.0, 300 mM NaCl, 0.5 M Imidazole, PMSF, Complete and 10% glycerol. PP17 was analyzed by SDS-PAGE (10%). Positive fractions were combined and dialyzed against TBS (20 mM Tris-HCl, pH-8, 150 mM NaCl) and stored at −80° C. until use. The protein concentration was determined by Bradford assay. The purification analysis is shown in FIGS. 4A and 4B.

### Example 2

### PCR Analysis of PP17 in Term Placenta

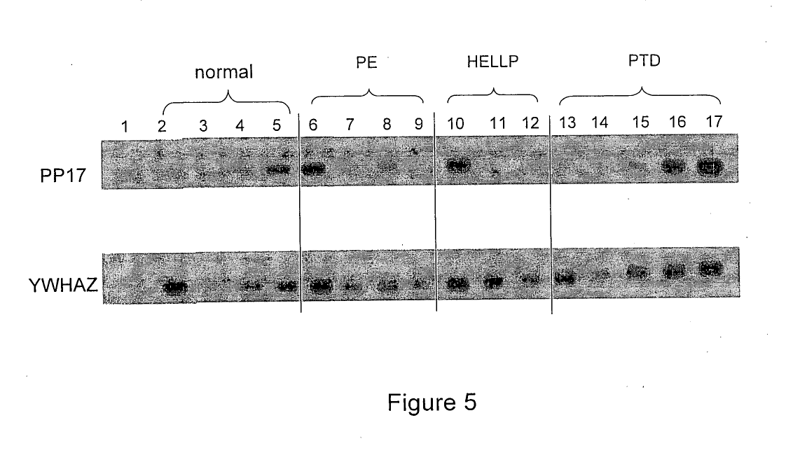

A total of 21 pregnant women participated in the study. Placental tissue was analyzed for the expression of PP17 by normal and real time PCR. Representative results of regular PCR are shown in FIG. 5. PP17 was expressed in the placenta of healthy controls and in the pathological cases.

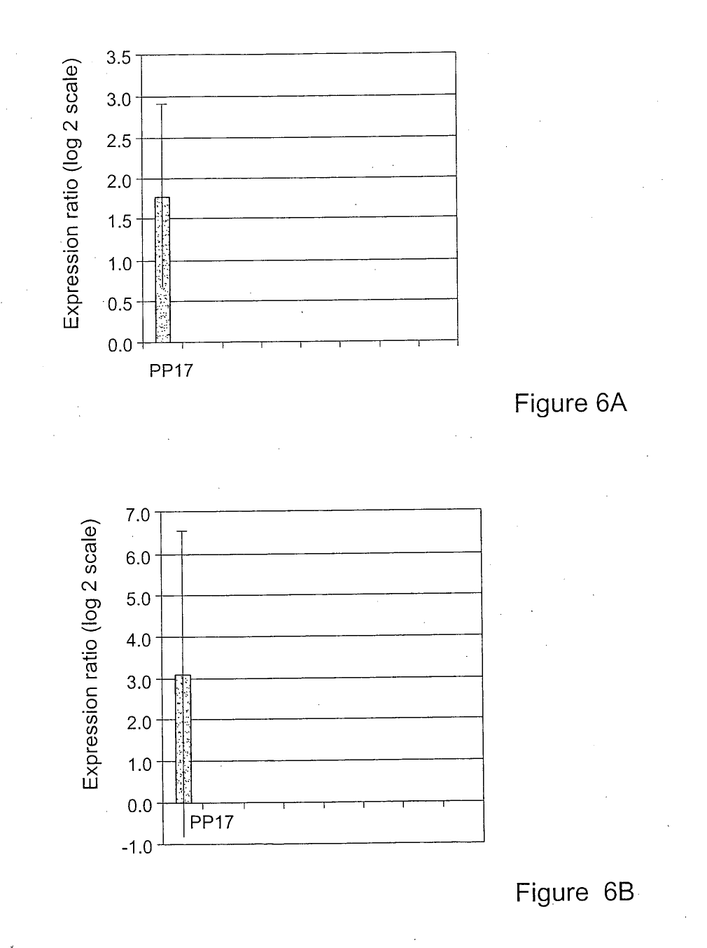

Quantification of PP17 expression was measured by real time PCR. The expression of PP 17 in placentas obtained from PE and HELLP women was compared to that of normal women. The results are shown in FIG. 6. PP17 was significantly (p=0.022) up-regulated by a factor of 3.073 in HELLP versus normal women, and was significantly (p=0.017) up-regulated by a factor of 1.783 in preeclampsia versus normal.

1. A method for diagnosing pregnancy-related complications in a pregnant woman, comprising:
(a) determining a level of Placental Protein 17 (PP17) in a bodily substance obtained from the pregnant woman; and
(b) comparing the determined level of PP17 to a standard level of PP17, a significant increase in the level of PP17 indicating the existence of a pregnancy-related complication in the pregnant woman.

2. The method of claim 1, wherein the bodily substance is selected from the group consisting of placental tissue and body fluids obtained from maternal blood, maternal saliva, maternal urine, amniotic fluid, umbilical cord blood, and chorionic villi.

3. The method of claim 1, wherein the level of PP17 is determined by measuring a level of PP17 DNA.

4. The method of claim 3, wherein the DNA is cDNA.

5. The method of claim 1, wherein the level of PP17 is determined by measuring a level of PP17 RNA.

6. The method of claim 5, wherein the PP17 mRNA expression level is measured.

7. The method of claim 1, wherein the level of PP17 is determined by measuring a level of PP17 protein.

8. The method of claim 7, wherein the level of PP17 protein is determined using an immunoassay.

9. The method of claim 1, wherein the pregnancy-related complication is preeclampsia.

10. The method of any of claim 1, combined with at least one additional method for diagnosing pregnancy-related complications in a pregnant woman.

11. A kit for diagnosing pregnancy-related complications in a pregnant woman, comprising:
(a) a first antibody which specifically binds to PP17;
(b) a second antibody which specifically binds to PP17 linked to a signal-generating molecule; and
(c) PP17 standard solutions.

12. A kit for diagnosing pregnancy-related complications in a pregnant woman, comprising:
(a) specific PP17 primers; and
(b) positive and negative cDNA controls.
